(19) United States
(12) Patent Application Publication
(10) Pub. No.: US 2024/0415852 A1
(43) Pub. Date: Dec. 19, 2024

(54) ORAL CODELIVERY OF NAVITOCLAX AND Bcl2 siRNA TREATING GASTIC CANCER

(71) Applicant: Board of Regents, The University of Texas System, Austin, TX (US)

(72) Inventors: Md Nurunnabi, El Paso, TX (US); Humayra Afrin, El Paso, TX (US); Raj Kumar, El Paso, TX (US)

(21) Appl. No.: 18/742,746

(22) Filed: Jun. 13, 2024

Related U.S. Application Data
(60) Provisional application No. 63/507,935, filed on Jun. 13, 2023.

(51) Int. Cl. A61K 31/635; A61K 9/00; A61K 9/16; A61P 35/00; C12N 15/113

(52) U.S. Cl. CPC A61K 31/635; A61K 9/0053; A61K 9/1617; A61K 9/1652; A61P 35/00; C12N 15/1135; C12N 2310/14

(57) ABSTRACT

Compositions and methods are described for oral treatment of gastric cancer. A composition of matter for treatment of gastric cancer includes navitoclax; and Bcl2 siRNA. The composition can include β-glucan and docosahexaenoic acid as a mucoadhesive nanocarrier.

### CROSS REFERENCE TO RELATED APPLICATION

Referring to the application data sheet filed herewith, this application claims a benefit of priority under 35 U.S.C. 119(e) from co-pending provisional patent application U.S. Ser. No. 63/507,935, filed Jun. 13, 2023, the contents of which are incorporated herein by reference in their entirety for all purposes.

### STATEMENT AS TO RIGHTS TO INVENTIONS MADE UNDER FEDERALLY-SPONSORED RESEARCH OR DEVELOPMENT

This invention was made with government support under Award No R03OD032624 awarded by National Institutes of Health (NIH). The government has certain rights in the invention.

### BACKGROUND INFORMATION

### 1. Field

The present invention relates generally to the field of medicine and disease treatment. More particularly, it concerns methods and compositions for oral therapy of stomach cancer.

### 2. Background

Gastric cancer is the second most leading cause of cancer related deaths worldwide and the only available treatment strategies are chemotherapy, immunotherapy, radiotherapy, and surgery. 5-Fuoro Uracil (5-FU), Capecitabine, Carboplatin, Cisplatin, Docetaxel, Epirubicin, Irinotecan, Oxaliplatin, Paclitaxel, Trifluridine and Tipiracil are the most used chemotherapeutics, and PDL1 inhibitors such as pembrolizumab, nivolumab are used in immunotherapy. Genes including IL-8, CLDN1, KRT17, DLSN7, and MMP7 are upregulated while GPER, KIAA1324, ADA, and SLC9A22 are down regulated in gastric cancer. Other genes such as STAB1, STAT3, Bcl-2 and Bcl-xl induce gastric cancer.

Heretofore, the requirements of attenuating stomach cancer while avoiding side effects and other undesirable consequences have not been fully met. In view of the foregoing, there is a need in the art for a solution that simultaneously solves all of these problems.

### SUMMARY

Gastric cancer is a challenging illness to treat due to the lack of early-stage diagnostic technology and targeted delivery systems. Currently, the only available treatments for gastric cancer are surgery, chemotherapy, immunotherapy, and radiation. These strategies are invasive and require systemic delivery that can damage healthy tissue. By creating a targeted delivery system to the stomach, gastric cancer can be treated in the early stages and reduce the negative effects to the rest of the body. With this in mind we developed a mucoadhesive nanocarrier composed of β-Glucan and Docosahexaenoic Acid (GADA) for controlled drug/gene delivery. Herein, we investigated the therapeutic potential of Bcl2 inhibitor Navitoclax (Navi) and siRNA (Bcl2) gene co-delivery using GADA nanocarrier. The therapeutic potential of the formulation was investigated against gastric cancer mice model. Higher Bcl2 inhibition efficacy was observed in western blotting and Tunnel assay in mice treated with GADA/Navi/Bcl2 siRNA nanoparticles compared to Navi/siRNA. Histology (H&E) and immunohistochemistry (Ki67, TUNEL, and BCl2) analysis confirmed significant reduction of the tumor region. GADA interaction with mucin led the drug release over 6 hours. In conclusion, the developed nanocarrier GADA is a prospective system for controlled delivery of drug and gene for gastric cancer therapy. Navitoclax and siRNA showed highly improved therapeutic efficacy with minimal side effects compared to conventional treatment. This study opens a new window for effective oral delivery of drug and gene for gastric cancer and other diseases.

Targeted oral delivery of anticancer treatments enhances the therapeutic effect by increasing retention time and therefore increase the drug concentration at the cancer site. Navitoclax is a Bcl2 inhibitor that acts by preventing the Bcl2 protein from halting cellular apoptosis, making it a good choice as a treatment against gastric cancer. Due to its hydrophobic properties, it was limited in its therapeutic use, however loading it onto a nanocarrier eliminates this issue allowing it to be used for oral delivery. We also hypothesized that a Bcl2 siRNA inhibitor could be used to knockdown the Bcl2 gene and thereby allow apoptosis of cancer cells. With this in mind, an oral co-delivery treatment was developed to produce a synergistic effect of Bcl2 gene silencing using siRNA and Bcl2 protein inhibitor Navitoclax to induce cell death of gastric cancer cells. To overcome the various limitations of oral delivery, we created a nanocarrier composed of BG which is a highly biocompatible polysaccharide and DHA a fat molecule, to form GADA. The results confirmed that GADA protected Navitoclax and siRNA from the harsh stomach environments, but also provided stability to the formulation, thus enhancing the retention time in the stomach for at least 5 hours. Furthermore, it was observed that GADA/Navi/Bcl2 siRNA treatment downregulated Bcl2 expression and induced apoptosis of cancer cells. Reduction of Ki67 marker also observed after treatment and histological staining showed complete remission of the tumor region in animals treated with GADA/Navi/Bcl2 siRNA. After analysis of all results, we conclude that GADA nanocarrier based oral treatment for gastric cancer is more effective than free siRNA and Navi. Therefore, we will continue this study to understand the mechanism of action of Navi and Bcl2 siRNA combination. Overall, the work done here opens a new platform for successful oral siRNA and drug delivery and verifies this treatment as a potential candidate for biological therapeutics.

An illustrative embodiment of the present disclosure is a composition of matter for oral treatment of gastric cancer, comprising navitoclax; and Bcl2 siRNA.

Another illustrative embodiment of the present disclosure is a method of treating gastric cancer comprising administering to a mammal in need thereof a compound comprising navitoclax; and Bcl2 siRNA.

Another illustrative embodiment of the present disclosure is a method of orally treating disease comprising administering to a mammal in need thereof a compound comprising navitoclax; and Bcl2 siRNA.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates a graphic abstract in accordance with an illustrative embodiment.

FIG. 2 illustrates schematic of Bcl2 inhibition and knockdown mechanism of action of Navitoclax and siRNA loaded GADA nanoparticles in accordance with an illustrative embodiment.

FIGS. 3A-3E illustrate (a) Particle size and (b) zeta potential and (c) FESEM images of GADA/Navi/Bcl2 siRNA. (d) SPR spectra of GADA with mucin and (e) cellular uptake confocal images of AGS cells treated with GADA/Navi/Bcl2 siRNA nanoparticles (scale bar=50 μm) in accordance with an illustrative embodiment.

FIGS. 4A-4D illustrate (a) preparation of 240 ppm NMNNU and (b) development of gastric cancer mice model. (c) photographs ((i) healthy control, (ii) untreated, (iii) GADA/Bcl2 siRNA, (iv) GADA/Navi, (v) Navi/Bcl2 siRNA and (vi) GADA/Navi/Bcl2siRNA) and weight of stomach harvested from animals treated with different formulation in accordance with an illustrative embodiment.

FIGS. 5A-5D illustrate (a) Immunohistochemistry for Bcl-2 protein. The scale bar represents 100 μm. (b) Quantification of Bcl2 immunofluorescence data analysis was presented. (c) Bcl2 western blot and (d) quantitative analysis of western blot in accordance with an illustrative embodiment.

FIG. 6 illustrates Histological staining (H&E) of stomach tissue. (Magnification 20×) in accordance with an illustrative embodiment.

### DETAILED DESCRIPTION

The disclosure of this application is technically related to co-pending U.S. Ser. No. ______ (attorney docket number UTEP2023-013-1), filed Jun. 13, 2024, the entire contents of which are hereby expressly incorporated by reference for all purposes.

Embodiments of this disclosure include a novel oral drug delivery vehicle that can facilitate oral delivery of anticancer drug, enhance retention duration within stomach to maintain therapeutic dose concentration within the cancer area, and thereby enhance cancer treatment with minimal toxicities/side effect. Embodiments of this disclosure can solve problems related to stomach cancer treatment. Heretofore, there are few technologies for oral medications for stomach cancer treatment. Embodiments of this disclosure can provide benefits for individual who are scared of needles because embodiments of this disclosure can include a painless oral medication approach. Embodiments of this disclosure will be beneficial for every stomach cancer patient who would otherwise go through chemotherapy and experience tremendous toxicities.

Medications according to embodiments of this disclosure can be administered orally instead of injection. The medication can reach the cancer site within a very short period of time as administered orally, and more importantly the medication can maintain retention and concentration within stomach for at least 6 hours. Consequently, a lesser dose will provide higher therapeutic effect and thereby we will be able to minimize anticancer drug mediated toxicities for other organs including heart, lung, and liver. Embodiments of this disclosure can include co-delivery of 2 individual modalities, for instance navitoclax in combination with siRNA. This combination delivery approach will provide synergistic therapeutic effect that is better than the single modalities.

B-cell lymphoma 2 (Bcl2) is crucial in the development of cancer since it can act as an oncogene, and it inhibits cell death as opposed to cell proliferation. One mechanism of cellular apoptosis is through mitochondrial caspase activation where the mitochondrial outer membrane is permeabilized, provoking the release of cytochrome c to bind with caspase-9-activating complex along with protease-activating factor-1 ultimately forming a heptameric activating complex. Bcl2 regulates the permeabilization of mitochondrial outer membrane and inhibits the release of caspase activating factors, thus preventing cell death from occurring. The Bcl2 family contains members of both pro-survival which are up-regulated and pro-apoptotic that are downregulated in various cancer types. The ability of Bcl2 to express opposing actions is thought to play a part in tumor formation as well as anticancer resistance of cancer cells. Overexpression of prosurvival Bcl2 protein leads to proliferation of cancer cells and apoptosis of healthy cells; to overcome this, Bcl2 levels need to be controlled. Therefore, directly targeting the apoptotic mechanism with a Bcl2 inhibitor could be a potential pathway to explore. The method of targeting this apoptotic pathway has yet to be explored due to various challenges, however, we believe it can be a potential treatment strategy for gastric cancer. There are two pathways to control Bcl2 overexpression; first is downregulation of the gene encoding Bcl2 and second is inhibiting the activity of Bcl2 protein to restore apoptotic processes. Bcl2 siRNA was selected to downregulate Bcl2 expression and Navitoclax was selected for Bcl2 inhibition activity and restore the apoptotic processes (Scheme 1). Therefore, we envision that a co-delivery of Bcl2 inhibitor and Bcl2 siRNA will synergistically accelerate the therapeutic outcome.

However, there are various obstacles for transportation and transfection with oral delivery of Bcl2 siRNA and Navitoclax including intense negative charge, large molecular weight, enzymatic degradation, variation of pH, harsh gastric fluid, and enzymatic degradation. Multiple conventional drug delivery systems exist; however, they lack the ability to deliver sufficient drug concentration directly to the site of the disease. Other issues with these conventional strategies are the negative side effects on healthy tissue resulting in systemic toxicity. Furthermore, poor membrane permeability, enzymatic degradation, thick mucus membrane, and off-target localization can decrease the bioavailability of the drug requiring higher drug concentrations to mitigate this loss during transport through the gastrointestinal tract. To overcome these limitations, controlled release and local oral delivery systems need to be developed. In last two decades nanotechnology-based drug delivery systems offered a variety of controlled and local oral drug delivery systems including pH responsive delivery, ROS responsive delivery, floating delivery, and polymeric delivery and mucoadhesive delivery systems. Among these recently discovered techniques that has more potential for oral delivery is mucoadhesive delivery system; it has strong interactions with mucin that increase retention time while also allowing a higher concentration of drugs to pass through the intestinal membrane. Conventional mucoadhesive nanocarriers are typically made with mucoadhesive polymers such as chitosan, alginate, pectin, poly(acrylic acid) and methyl cellulose. These mucoadhesive nanocarriers are limited in their potential for oral delivery for gastric cancer treatment due to toxicity, low stability, enzymatic degradation, toxic degradation processes, toxic monomers aggregation, and residual material associated with them. Another limitation of some drugs is the occurrence of local ulcers due to prolonged contact with a drug possessing ulcerogenic properties. To help mitigate these limitations we believe an effective nanocarrier for the delivery of Bcl2 siRNA and Navitoclax needs to be developed.

Herein, we developed a mucoadhesive nanocarrier GADA composed of 3-glucan (BG) (a carbohydrate polymer) and DHA (Docosahexaenoic acid) to achieve an effective targeted therapy for gastric cancer treatment. Embodiments include the preparation of nanoparticles of GADA loaded with Navitoclax and siRNA and the delivery mechanism for gastric cancer therapy. We hypothesized that GADA could protect Navitoclax and siRNA from the harsh conditions in the stomach. To evaluate the efficacy of the nanocarrier, we evaluated the physical characterization, and cellular uptake/internalization with in vitro studies as well as in vivo analysis of therapeutic affect against gastric cancer mice model. Toxicity was also evaluated for safety of the treatment with blood serum analysis. Overall, the results will demonstrate this oral delivery approach may provide a safer and more effective option for gastric cancer treatment in the clinic.

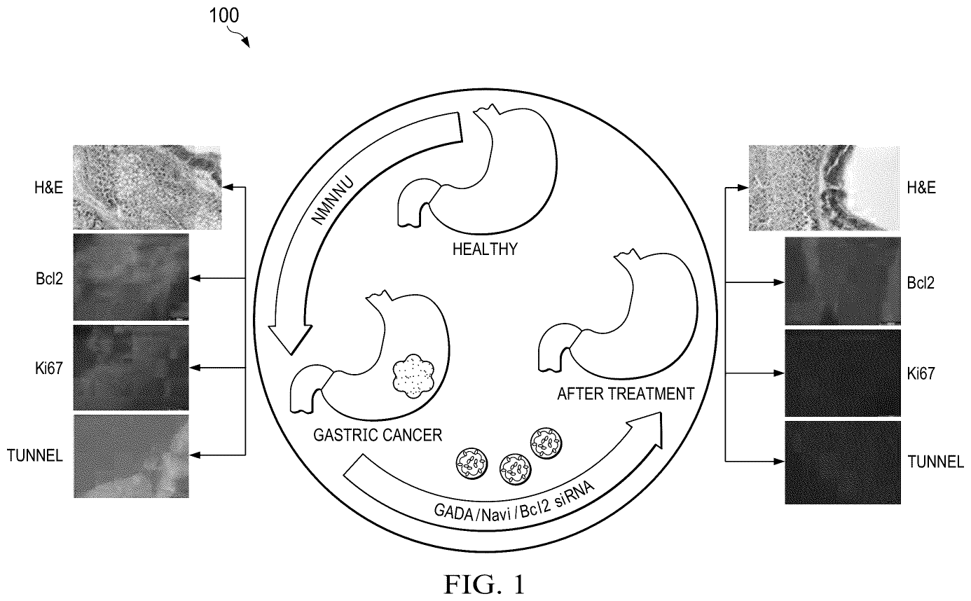

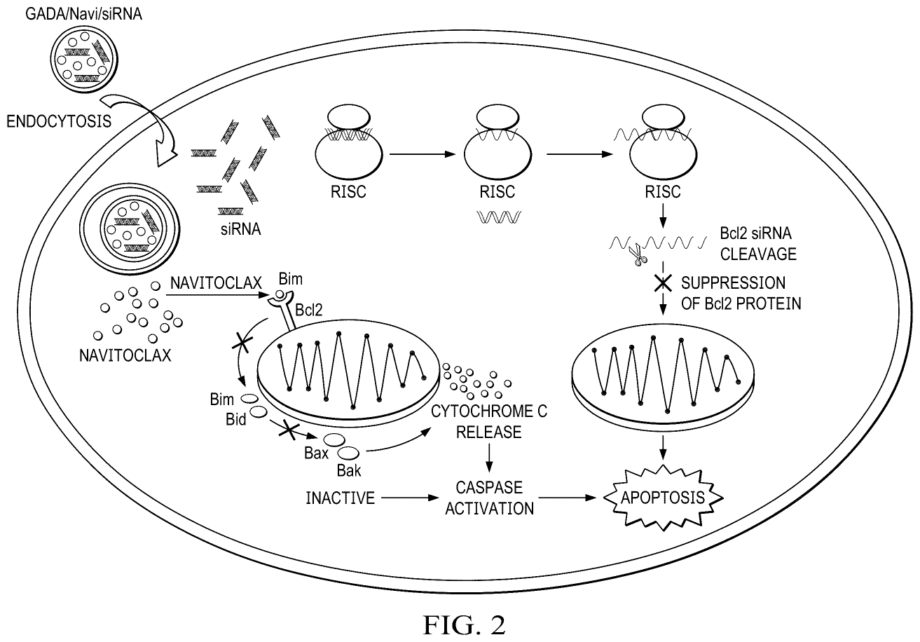

FIG. 1 shows a graphical abstract 100 of embodiments of this disclosure. FIG. 2 shows a schematic of Bcl2 inhibition and knockdown mechanism of action of Navitoclax and siRNA loaded GADA nanoparticles.

### Examples

Specific exemplary embodiments will now be further described by the following, nonlimiting examples which will serve to illustrate in some detail various features. The following examples are included to facilitate an understanding of ways in which embodiments of the present disclosure may be practiced. However, it should be appreciated that many changes can be made in the exemplary embodiments which are disclosed while still obtaining like or similar result without departing from the scope of embodiments of the present disclosure. Accordingly, the examples should not be construed as limiting the scope of the present disclosure.

### Materials and Methods

### Materials

Deionized water (resistivity of 18.2 MΩ), used for all experiments, was obtained from an in-lab Milli-Q® IQ 7000 Ultrapure Water System (EMD Millipore, Bedford, MA, USA). β-glucan (Barley, Low Viscosity) was purchased from Neogen, USA. DHA was purchased from Millipore Sigma, USA. Phosphate buffered saline (PBS) (pH 7.4) was procured from Fisher Chemicals (Fair Lawn, NJ, USA). Cell culture reagents, including F-12K Medium, fetal bovine serum (FBS), Dulbecco's PBS, penicillin/streptomycin, and trypsin, were purchased from Gibco BRL (Carlsbad, CA, USA). Hanks' Balanced Salt solution was purchased from Merck. CyQUANT MTT Cell Proliferation Assay Kit (V13154), Click-iT™ TUNEL Alexa Fluor™ 488 Imaging Assay, for microscopy & HCS (C10245) were from Thermo Fisher. Anti-BCL-2 and anti-β-actin antibodies were purchased from Abclonal. The silica wafer and stub were purchased from Ted Pella, Inc., USA.

### Synthesis of β-Glucan-DHA

GADA was synthesized using 1:15 molar ratio as reported in our previous publication. Initially, in a 20 mL clean glass vial, 10 mg β-glucan was added to 500 μL of water and stored at 50° C. for 20 min. To the transparent β-glucan solution we added 15 moles of DHA dropwise, with respect to BG. It is then stirred over night at room temperature; the resulting solution is cloudy. Following this, free DHA in the supernatant was removed through centrifugation at 1000 rpm for 8 min. The collected GADA was resuspended in 500 μL for water for further use.

### Formulation of Nanoparticles

Initially, a solution of siRNA (1 μM) and Navitoclax (3 mM) in DMSO:PBS (6:4) was prepared. Then siRNA and Navitoclax was loaded into GADA nanocarrier through drop wise addition of siRNA and Navitoclax solution into BG-DHA solution (1 mg/mL) under stirring. The nanoparticles were collected by centrifugation and freeze drying. The prepared nanoparticles were stored at 4° C. for future use. GADA nanoparticles were also prepared through dropwise addition of GADA solution in PBS into acetone under stirring. The three nanoparticle solutions are represented as GADA/Bcl2 siRNA (siRNA loaded only), GADA/Navi (navitoclax loaded only), and GADA/Navi/Bcl2 siRNA (Navi and Bcl2 siRNA loaded).

Ranges of siRNA concentration would go from 10, 20, 40, 60, 80, 100 nm. Dosage ranged from 1, 3, 5, 10, 20 mg/kg of body weight of mice.

### FESEM

Accurate particle size and morphology of samples was also observed using an FESEM (Hitachi S-4800 series II cold field emission SEM). The dispersion of nanoparticles in water was drop-coated on a silica wafer and dried at room temperature. The dried silica wafer was transferred onto a carbon tape applied on a clean SEM stub, which was subsequently sputter-coated with gold for 60 second before the FESEM observations.

### Stability of the Particle

Nanoparticle stability was studied to determine the capability of the GADA nanocarrier to protect loaded Navitoclax and siRNA. The stability of nanoparticles was evaluated at room temperature (RT) and 4° C. The nanoparticles are dispersed in water, stored at room temperature and 4° C., and observed over a 60 day period. On day 2, 10, 20, 30, 45, and 60 the size and zeta potential of nanoparticles was measured using DLS to monitor the stability.

For oral formulations, it is necessary to determine the stability of the nanoparticles in the stomach environment. Hence, we also studied the stability of nanoparticles in PBS and simulated gastric fluid (SGF). The nanoparticles are dispersed in PBS and SGF and stored at room temperature. The size and zeta potential were measured during a 6 hr period at 30 min, 1 hr, 2 hr, 4 hr, and 6 hr, using DLS to evaluate the stability in PBS and SGF.

### Drug Loading and Drug Encapsulation Efficiency

The drug loading (DL %) and drug encapsulation efficiency (EE %) of GADA/Navi/Bcl2 siRNA formulation was evaluated using a plate reader. The known amount of the formulation was dispersed in an organic solvent and sonicated. The concentration of drug was quantified using absorption. The drug loading and encapsulation efficiency were calculated using following equations.

$$EE \% = \frac{Amount of drug present in the formulation}{Amount of drug using in formulation} \\ DL \% = \frac{Amount of drug present in the formulation}{Amount of formulation}$$

### Surface Plasmon Resonance (SPR)

The physicochemical interactions between GADA and mucin were studied using SPR (ibClu, South Korea) technique. We used GADA as a ligand over carboxylic acid functionalized gold chip. 1×PBS was used as running buffer and 50 μM mucin as analyte. The experiment was conducted according to manufacturer's instructions. Initially, EDC and NHS were added to initiate activation. Then GADA was introduced to observe the interaction, and finally to end the experiment blocking buffer was added. The response peak data was collected and made a graph in Prism GraphPad.

### Cytotoxicity

The cytotoxicity of the nanoparticle formulation was studied using adenocarcinoma gastric cell line (AGS). Cells were grown in F-12K media supplemented with 20% fetal bovine serum at 5% CO₂ humidified atmosphere. Cells were harvested using trypsin and 1×10⁴ cells with 100 μL of serum free media per well was plated in 96-well plate and incubated for 24 and 48 h. At end of time interval, media was removed and known concentrations of samples 1×PBS, Navi, GADA/Navi, GADA/Navi/Bcl2 siRNA, GADA/Bcl2 siRNA, Navi/Bcl2 siRNA, (Navi 10, 20, 30, 40, and 50 μM and Bcl2 siRNA 20, 40, 60, 80, and 100 nM) and GADA were added to the cells. The plate was then incubated for 24 h and 48 h. Cell viability was measured using MTT cell proliferation assay kit according to manufacturer's instruction. The absorbance of MTT was measured by using a SYNERGY H1 microplate reader (BioTek) at a wavelength of 570 nm and subtracted the background absorbance recorded at 630 nm.

### Development of Gastric Cancer Mice Model

To create a gastric cancer mouse model, animals were treated with N-methyl-N-nitrosourea (NMNNU) in drinking water (IACUC protocol no. A-201910-A). We used 6-week-old C57BL/6 mice in 12 hr light and 12 hr dark cycle. The mice were treated with 240 ppm NMNNU on weeks 1, 3, 5, 7, and 9. On week 10, mice used for study. A total of 30 mice were divided into 6 groups: control (healthy), untreated, Navi/siRNA, GADA/Navi, GADA/Bcl2 siRNA, and Navi/Bcl2 siRNA GADA/Navi/Bcl2 siRNA. To study the therapeutic potential of formulations, 100 μL of formulation containing Navi (3 mg/Kg) and/or siRNA (100 nM) was administrated orally. All the formulation administrated on day 1, 4, 7, 10, and 13 and were freshly prepared before oral administration. Animal sacrifices were performed on the 3^{rd} day after last treatment and major organs harvested and blood collected in Vacutainer® Plus Plastic Heparin Blood Collection Tubes with Lithium Heparin for Plasma Determination for further studies. The weight and size of the stomach was measured using a ruler followed by imaging. All the tissues and major organs were stored in liquid nitrogen. Blood was stored at −80° C.

### In Vivo Protein Expression

The tissue was homogenized with glass motor and pestle over dry ice with RIPA buffer containing a protease inhibitor. The homogenate was centrifuged, and the supernatant was collected. The protein concentration was calculated using Bradford's assay (Sigma-Aldrich). For each protein sample 30 μg was used to perform SDS-PAGE (10%) at 80 V for 4 hr at RT. Using a transfer unit, proteins were transferred to a PVDF membrane at 25 V overnight at 4° C. PVDF membrane was blocked by incubation in 3% BSA containing TBST for 90 min. It was then incubated with Bcl2 primary antibody for 2 hr and washed with TBST 3 times for 5 min each. This is followed by incubation with secondary antibody for 1 hr and washed with TBST 5 times for 5 min each. After, the membrane is treated with 3-actin primary antibodies (Abclonal, USA) overnight at 4° C. followed by washing with ice-cold PBS and incubated with horseradish peroxidase-conjugated secondary antibody (Abclonal, USA) for 120 min at RT. Manufacturer's protocol was used for dilutions. After treatment with ECL mixture, the bands were visualized using a ChemiDoc imaging system (BioRad, USA). Image J software was used to quantify the band intensities.

### Results and Discussion

### Particle Size and Zeta Potential

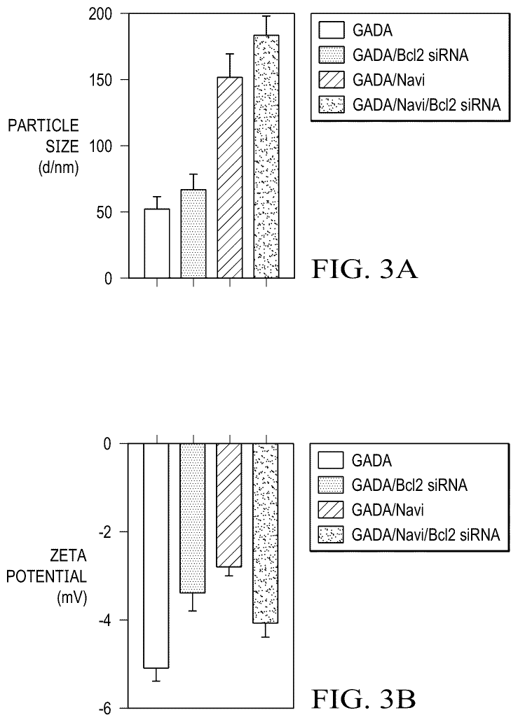

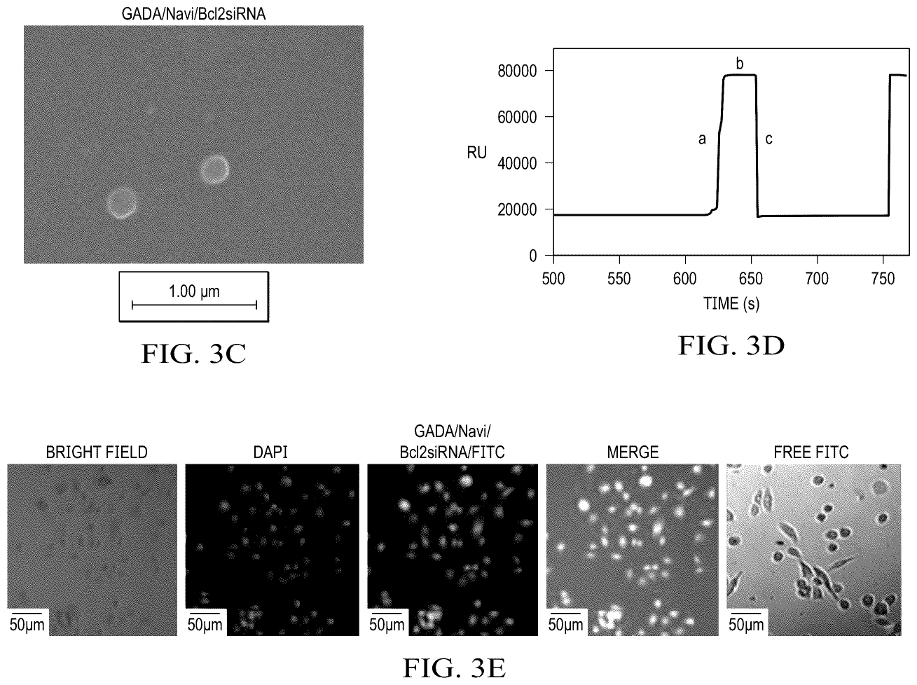

By stirring DHA and BG overnight, GADA is formed through hydrogen bonding between the hydroxyl group of BG and the carboxylic acid of DHA. Dropwise addition of Navitoclax and siRNA solution into β-glucan-DHA solution under stirring leads to formation of nanoparticles immediately as we detected nanosized particles by DLS after injection. We continue stirring up to 5 minutes to achieve complete mixing which yields the smaller size nanoparticles with narrow size distribution. The particle size and zeta potential of all the nanoparticles was measured. Particle size and zeta potential of formulated nanoparticles are shown in FIG. 3A and FIG. 3B. The size of the loaded nanoparticles is higher compared to bare nanoparticles. A comparison of siRNA loaded nanoparticles to Navi loaded nanoparticles, shows the Navi nanoparticles are slightly bigger. Among all the nanoparticles, GADA/Navi/Bcl2 siRNA nanoparticles are larger in size. The size of the nanoparticles ranges from 52±9 nm for GADA nanoparticles to 183±12 nm for GADA/Navi/Bcl2 siRNA. It can be concluded that all the nanoparticles formulated are under 200 nm.

Zeta potential measures the charge stability of a particle's dispersion. Zeta potential indicates the degree of repulsion between adjacent, similarly charged particles in dispersion. Zeta potential between −30 mV to +30 mV indicated a good dispersion of charge stability. We measured the zeta potential of nanoparticles dispersion in water. All nanoparticles showed negative zeta potential. The nanoparticles GADA, GADA/Navi, GADA/Bcl2 siRNA, and GADA/Navi/Bcl2 siRNA showed zeta potential −5.1±0.3, −3.4±0.4, −2.8±0.2, and −4.1±0.3 mV respectively. There is no significant difference in zeta potential because all particles are GADA and water except those loaded with Navitoclax, siRNA, and Navi/siRNA. The slight difference in charge could be due to the presence of free Navitoclax or siRNA in solution or on the surface of the nanocarrier.

### FESEM

Initially, particle size was measured using DLS, however it measured a size that was larger than the actual particle size due to DLS measuring hydrodynamic size of particles. To determine the actual size of the nanoparticles FESEM was used. FIG. 3C represents the FESEM image of GADA/Navi/Bcl2 siRNA nanoparticles. The size of GADA/Navi/Bcl2 siRNA nanoparticles using DLS measurement is 183±12 nm, however when measured using FESEM the particle size was determined to be ˜150 nm with a spherical shape. Li et al. concluded for oral drug delivery nanoparticles size of 100 nm or less are promising over larger sizes. Banerjee et al. investigated the size of the nanoparticles on cellular uptake in oral drug delivery and confirmed smaller particle size increases cellular uptake. Since the size of our formulated nanoparticles are less than 200 nm, we believe that they are suitable as treatment for gastric cancer by oral drug delivery.

### Stability

The storage stability of nanoparticles is very important as more stable drugs can significantly reduce the cost of transportation and storage. Nanomedicine, which typically requires cold chain storage is usually expensive and difficult to transport and maintain in remote locations. So, formulation with room temperature stability has great market value. Hence, we studied the stability of nanoparticles at room temperature and 4° C. for 60 days. Altogether it is concluded that the prepared nanoparticles are stable in PBS as well as SGF.

### Drug Loading and Drug Encapsulation

The drug loading and drug encapsulation into the GADA/Navi/Bcl2 siRNA was quantified measuring the amount of drug present in the formulation. GADA/Navi/Bcl2 siRNA nanoparticles showed 92% of drug loading efficiency. The high loading efficiency may be due to the mucoadhesive nature of GADA allowing interaction with the nanoparticles leading to higher drug retention. Other optimum experimental parameters may also favor the high drug loading efficiency. The encapsulation efficiency of the drug in GADA/Navi/Bcl2 siRNA was found as 22%. These results indicate that using GADA as a nanocarrier enhances the drug loading capacity and encapsulation efficiency of Navi and Bcl2 siRNA. Based on this we also believe that GADA will be suitable as a carrier for other oral drugs.

### Cellular Uptake

Fluorescence microscopy was used to investigate the cellular uptake potential of GADA nanocarrier. Results presented in FIG. 3E show the nanocarrier containing FITC was internalized into AGS cells. The AGS cells exhibited strongest green fluorescence after treatment with the GADA/FITC/Navi/Bcl2 siRNA compared to free FITC after 4 hr of treatment. In summary, GADA nanocarrier effectively delivered the Navitoclax and siRNA into the cells and into the nucleus. Hence, GADA nanocarrier is a prospective choice for oral delivery for gastric cancer therapy.

### Mucoadhesive Properties of BG-DHA

Mucin is key component of mucus layer therefore it plays a key role in the development of oral formulations. The interaction between GADA and mucin was studied using SPR analysis. This study establishes the binding efficacy of GADA nanoparticles with mucin present in the stomach including with acidic simulated gastric juice. Initially gold chip functionalized with carboxyl functional group modified β-glucan and mucin solutions. The strong binding efficacy was confirmed through peak response more than 80000 RU from baseline. In FIG. 1D, peak “a” represents GADA and mucin association, “b” indicates plateau as it continues binding, and curve “c” indicates dissociation of GADA with mucin. Since GADA has a binding interaction with mucin it can be established as a mucoadhesive nanocarrier. Mucoadhesive nature of nanocarriers enhances the retention time in the stomach and increases the absorption through epithelium.

FIG. 3A shows particle size. FIG. 3B shows zeta potential. FIG. 3C shows FESEM images of GADA/Navi/Bcl2 siRNA. FIG. 3D shows SPR spectra of GADA with mucin. FIG. 3E shows cellular uptake confocal images of AGS cells treated with GADA/Navi/Bcl2 siRNA nanoparticles (scale bar=50 μm).

### Cell Viability

The mitochondrial enzymes are responsible for reducing the tetrazolium salt (MTT) into purple formazan crystals which is indicator of cell viability/cytotoxicity. The therapeutic effect of formulations was carried out by quantifying the cell viability using MTT assay. GADA nanocarrier shows negligible toxicity as it is composed of carbohydrate polymer β-glucan. Navitoclax cell viability was found to be 65.2±5.32% at end of 24 h, whereas the GADA/Navi showed cell viability 39.4±4.72%. This demonstrates GADA enhanced the effect of Navitoclax to kill cancer cells as the cell viability decreased by about 25 percent. The cell viability notably reduced in case of GADA/Navi/Bcl2 siRNA, and it was found that 28.3±6.32% indicating that the addition Bcl2 siRNA further enhances the therapeutic effect of the treatment. The cell viability data establishes co-delivery of Navitoclax and Bcl2 siRNA with GADA as the carrier reduces the viability of cancer cells therefore increasing the therapeutic affect while also maintaining safety to noncancerous cells.

### Phenotypical In Vivo Treatment Efficiency

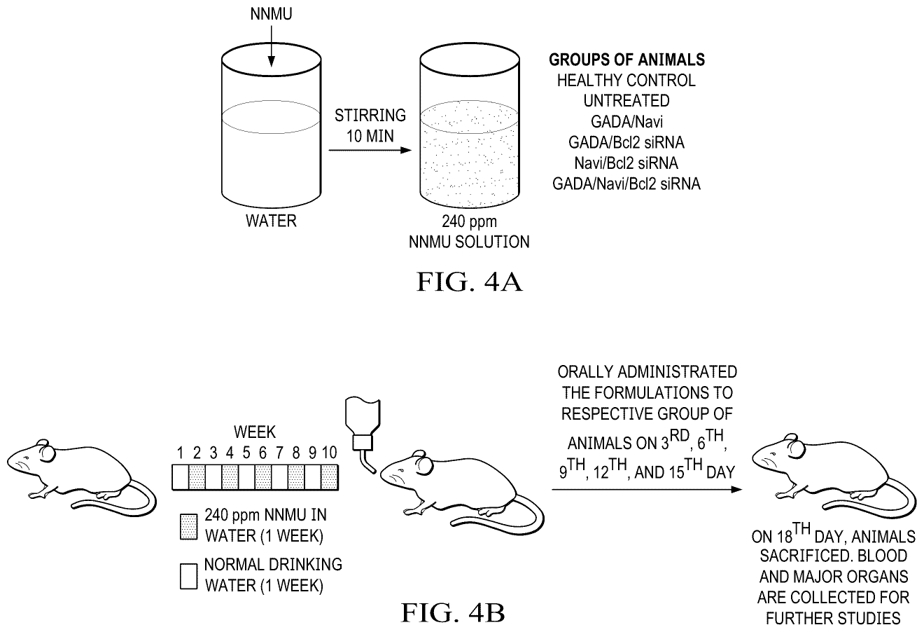

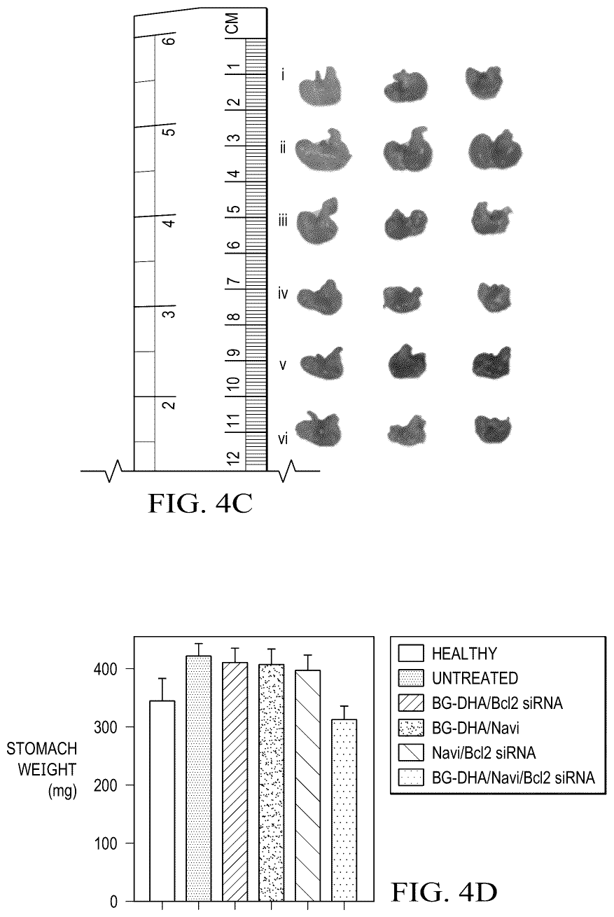

In-vivo study was conducted using a gastric cancer animal model; the schematic is represented in FIG. 4A and FIG. 4B. After the last treatment, we sacrificed the mice and collected the major organs and blood for further studies. Immediately after harvesting the major organs, we measured the weight of stomach and took the pictures of stomach along a measurement scale to determine the size. FIG. 4C and FIG. 4D show the stomach images and weight from the animal treated with different formulations. Five animals were used in each group. However, two stomach tissues were immediately processed for H&E staining, immunohistochemistry, and western blot analysis which is why each group only shows three stomachs. The stomach weight of the animals in the untreated group is reduced to the reduction of ghrelin production which decreases appetite. Based on the stomach weight for each treatment group, we can see the GADA/Navi/Bcl2 siRNA nanoparticles showed the stomach weight similar to the healthy control. The swelling observed in the stomach images of the untreated group is likely the result of buildup of ascites fluid caused by the gastric cancer.

FIG. 4A shows preparation of 240 ppm NMNNU. FIG. 4B shows development of gastric cancer mice model. FIG. 4C shows photographs of (row i) healthy control, (row ii) untreated, (row iii) GADA/Bcl2 siRNA, (row iv) GADA/Navi, (row v) Navi/Bcl2 siRNA and (row vi) GADA/Navi/Bcl2siRNA). FIG. 4D shows weight of stomach harvested from animals treated with different formulation.

Oral Bcl2 siRNA Caused In-Vivo Gene Silencing and Apoptosis.

TUNEL assay was used to study the apoptosis of cancer cells within the stomach tissue collected from the animal groups including healthy, untreated, and treated with different formulation. From the imaging, we found significantly higher apoptosis in GADA/Navi/Bcl2 siRNA tissue samples compared to other treatments group such as GADA/Navi, GADA/Bcl2 siRNA, and siRNA/Navi. It can also be seen that the results of the untreated group are almost the same as the healthy control, but when GADA/Navi/Bcl2 siRNA nanoparticle treatment is compared to the untreated group it is evident that the treatment induces a higher amount of apoptosis of gastric cancer cells. Further, apoptosis was quantified using mean fluorescent intensity. The apoptosis percentages for healthy, untreated, GADA/Bcl2 siRNA, GADA/Navi, siRNA/Navi, and GADA/Navi/Bcl2 siRNA are respectively, 100, 99, 121, 109, 101, and 138%. The results of the apoptosis analysis conclude GADA/Navi/Bcl2 siRNA treated group induces more gastric cancer apoptosis compared to other groups.

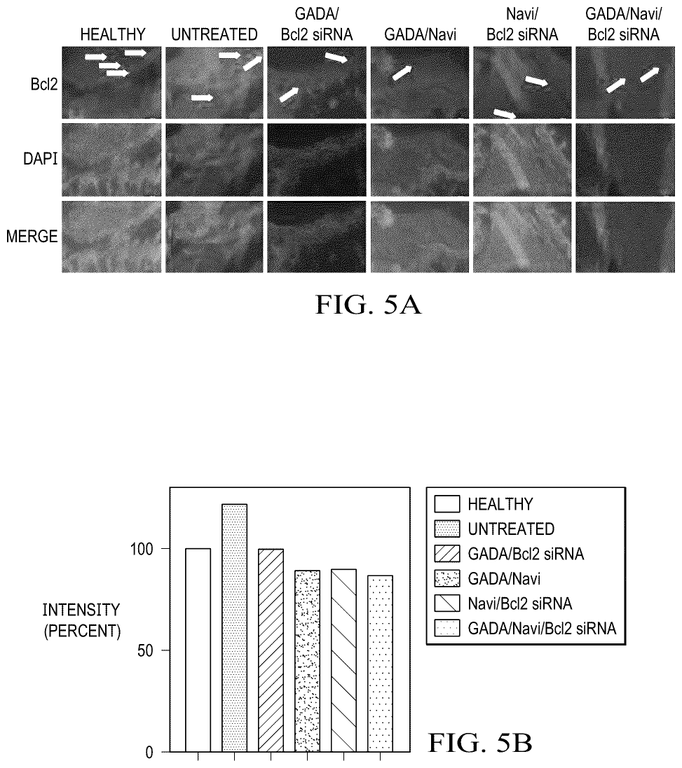

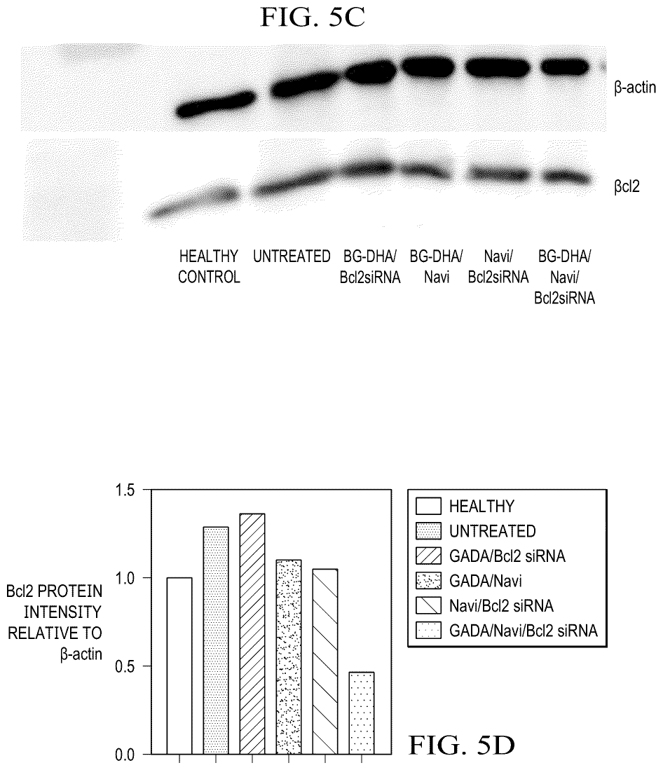

Bcl2 immunohistochemistry (IHC) of the stomach tissues collected from the animal was also studied and those results are presented in FIG. 5A. The relative Bcl2 expression on the tissue section was calculated considering the healthy mice Bcl2 expression as 100% shown in FIG. 5B. The immunohistochemistry results are reciprocated with WB results. The nanoparticles GADA/Navi/Bcl2 siRNA showed the lowest Bcl2 expression, whereas the untreated mice tissue exhibited highest Bcl2 expression. The relative expression of Bcl2 in animal group healthy, untreated, GADA/Bcl2 siRNA, GADA/Navi, Navi/siRNA, and GADA/Navi/Bcl2 siRNA nanoparticles are 100, 121, 100, 88, 89, and 86%, respectively. The western blot and immunohistochemistry results confirmed that GADA/Navi/Bcl2 siRNA nanoparticles has potential of higher Bcl2 inhibition efficiency concluding that Navi and siRNA co-delivery showed a synergistic effect in downregulating the Bcl2 gene.

We performed the western blot analysis to study if the apoptosis is due to Bcl2 protein downregulation. Western blotting was performed using the total protein collected from stomach of animals treated with the different formulations. The western blot results are shown in FIG. 5C, and quantification results shown in FIG. 5D. FIG. 5C and FIG. 5D confirmed that significant down regulation of Bcl2 gene in the group treated with GADA/Navi/Bcl2 siRNA nanoparticles. The relative expression of Bcl2 was 1, 1.28, 1.36, 1.1, 1.05, and 0.47, respectively for healthy, untreated, GADA/Bcl2 siRNA, GADA/Navi, Navi/siRNA, and GADA/Navi/Bcl2 siRNA. Western blot analysis confirms that GADA nanocarrier was protected from gastric condition and able to pass through mucus layer of the intestine and reach to the site of action to downregulate the Bcl2 gene.

FIG. 5A shows immunohistochemistry for Bcl-2 protein. The scale bar represents 100 μm. FIG. 5B shows quantification of Bcl2 immunofluorescence data analysis was presented. FIG. 5C shows Bcl2 western blot. FIG. 5D shows quantitative analysis of western blot.

### Therapeutic Efficacy In Vivo

Antitumor efficacy of prepared nanoparticles studied using immunofluorescence assay. Cancer cell nucleus contains the Ki67 protein which provides information about cell proliferation and gastric cancer prognosis making the Ki67 protein a key factor for analyzing chemosensitivity and therapeutic effects Using immunofluorescence assay, we measure the cell proliferation marker Ki67 of stomach tissue section of different treatment groups. Results that show higher levels of Ki67 protein correspond to lower chemosensitivity. Untreated group showed more Ki67 expression. However, group treated with GADA/Navi/Bcl2 siRNA nanoparticles showed significant reduction in Ki67 expression. Further, we quantified the Ki67 expression of the immunofluorescence images. The area with Ki67 positive cells was measured and compared to the whole tissue section with DAPI stained. The relative percent of area of different treatment groups such as healthy, untreated, and treated with GADA/Navi, GADA/Bcl2 siRNA, and GADA/Navi/Bcl2 siRNA are respectively, 100%, 107%, 80%, 69%, 87%, and 67%. Compared to healthy animals, the untreated group showed 7% higher Ki67 expression. The group of animals treated with GADA/Navi/Bcl2 siRNA nanoparticles showed 33% reduction of Ki67 expression compared to untreated gastric cancer model animals. The results demonstrate that the Ki67 expression is comparatively higher in animals treated with GADA/Navi and GADA/Bcl2 siRNA than the animals treated with GADA/Navi/Bcl2 siRNA.

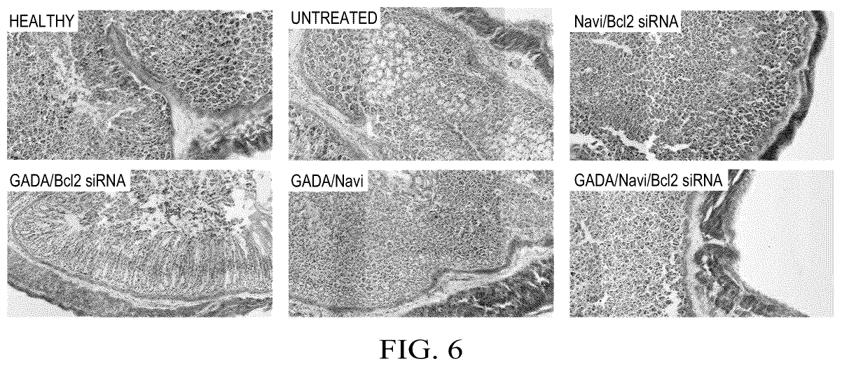

H&E staining of the stomach tissues was conducted to analyze the therapeutic effect of the treatment against gastric cancer and also the effect of inflammation to the surrounding cells caused by gastric cancer. The results of this are shown in FIG. 6 with images taken at 20× magnification. Indicated by the black arrow, presence of a tumor is seen in the untreated group, GADA/Navi, and siRNA/Navi. Notably, there is no visible tumor in the group treated with GADA/Navi/Bcl2 siRNA indicating that this nanoparticle has potential as a treatment against gastric cancer. FIG. 6 shows histological staining (H&E) of stomach tissue. (Magnification 20×).

### Toxicity

The blood collected from the animals was used to conduct serum biochemistry analysis to evaluate the toxicity of the nanoparticles testing several metabolic markers in the liver and kidney. The results are similar in range compared to the healthy control concluding that oral administration of the nanoparticles such as GADA/Navi, GADA/Bcl2 siRNA and GADA/Navi/Bcl2 siRNA are not toxic to liver and kidney.

Total blood count analysis was conducted to determine platelet (PLT), mean platelet volume (MPLT), red blood cells (RBC) and white blood cells (WBC). In literature, it was reported that Navitoclax induced platelet apoptosis, resulting in severe thrombocytopenia. Platelet survival and production depends on Bcl2-xL protein, a member of Bcl2 protein family. The results here do not indicate that the treatment induces platelet apoptosis due to GADA as the delivery vehicle and direct targeting to the site of gastric cancer. This analysis concludes that GADA is a safe nanocarrier for oral delivery of gastric cancer.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition of matter for treatment of gastric cancer, comprising:
navitoclax; and
Bcl2 siRNA.

2. The composition of claim 1, further comprising at least one mucoadhesive nanocarrier.

3. The composition of claim 2, wherein the at least one mucoadhesive nanocarrier comprises at least one carbohydrate polymer and at least one fat molecule.

4. The composition of claim 3, wherein the at least one carbohydrate polymer comprises at least one biocompatible polysaccharide.

5. The composition of claim 4, wherein the at least one biocompatible polysaccharide comprises at least one β-glucan.

6. The composition of claim 5, wherein the at least one fat molecule comprises at least one docosahexaenoic acid.

7. The composition of claim 3, wherein the at least one fat molecule comprises at least one docosahexaenoic acid.

8. A method of treatment of gastric cancer, comprising:
administering to a mammal in need thereof a compound including
navitoclax; and
Bcl2 siRNA.

9. The method of claim 8, wherein administering comprises oral administration.

10. The method of claim 9, wherein administering includes administering the compound wherein the compound includes at least one mucoadhesive nanocarrier.

11. The method of claim 10, wherein administering includes administering the compound wherein the at least one mucoadhesive nanocarrier comprises at least one carbohydrate polymer and at least one fat molecule.

12. The method of claim 11, wherein administering includes administering the compound wherein the at least one carbohydrate polymer comprises at least one biocompatible polysaccharide.

13. The method of claim 12, wherein administering includes administering the compound wherein the at least one biocompatible polysaccharide comprises at least one β-glucan.

14. The method of claim 13, wherein administering includes administering the compound wherein the at least one fat molecule comprises at least one docosahexaenoic acid.

15. The method of claim 11, wherein administering includes administering the compound wherein the at least one fat molecule comprises at least one docosahexaenoic acid.

16. A method of oral treatment of disease, comprising:
administering to a mammal in need thereof a compound comprising
navitoclax; and
Bcl2 siRNA.

17. The method of claim 16, wherein administering includes administering the compound wherein the compound includes at least one mucoadhesive nanocarrier.

18. The method of claim 17, wherein administering includes administering the compound wherein the at least one mucoadhesive nanocarrier comprises at least one carbohydrate polymer and at least one fat molecule.

19. The method of claim 18, wherein administering includes administering the compound wherein the at least one carbohydrate polymer comprises at least one biocompatible polysaccharide.

20. The method of claim 19, wherein administering includes administering the compound wherein the at least one biocompatible polysaccharide comprises at least one β-glucan.

21. The method of claim 20, wherein administering includes administering the compound wherein the at least one fat molecule comprises at least one docosahexaenoic acid.
